(19) United States
(12) Patent Application Publication
(10) Pub. No.: US 2017/0051243 A1
(43) Pub. Date: Feb. 23, 2017

(54) RECIRCULATING BIOREACTOR EXHAUST SYSTEM

(71) Applicant: Sarfaraz K. Niazi, Deerfield, IL (US)

(72) Inventor: Sarfaraz K. Niazi, Deerfield, IL (US)

(73) Assignee: THERAPEUTIC PROTEINS INTERNATIONAL, LLC, Chicago, IL (US)

(21) Appl. No.: 14/831,592

(22) Filed: Aug. 20, 2015

(51) Int. Cl. C12M 1/00; C12M 1/34; C12M 1/12; B01D 53/00

(52) U.S. Cl. CPC C12M 29/24; B01D 53/002; C12M 41/12; C12M 41/34; C12M 37/02

(57) ABSTRACT

A recirculating bioreactor exhaust apparatus designed to remove a gaseous element from the bioreactor exhaust, such as carbon dioxide, by freezing the gaseous element and then returning the remaining exhaust gas to the bioreactor and a method of use.

### BACKGROUND OF THE INVENTION

Bioreactors are devices that convert nutritional elements into useful products using biological catalysts (biological culture) such as human and animal tissue cells, microorganism, baculoviruses and insect cells, etc. The main function of a bioreactor is to provide a controlled environment for the growth of the biological catalysts to obtain a desired product. A large number of designs of bioreactors are available as evidenced by hundreds of patents issued to specific design elements of bioreactors. A variety of vessels and methods have been developed over the years to carry out chemical, biochemical and/or biological processing.

Bioreactor systems and related process improvements would be useful in the preparation of a variety of products produced from a biological source. One such improvement is the environment where a bioreactor may be operated, for example, an ISO 14644 Class 9 area, to reduce the cost of such facilities as currently needed for the production of these products. Currently, clean rooms are required to prevent contamination of the bioreactors in a facility and to prevent contamination of the clean rooms themselves from the content of the bioreactor that are inevitably carried in the exhaust gases. To maintain clean rooms, the bioreactor exhaust gas is filtered using, e.g., sterilizing filters, but this step adds substantial cost as well as creating complexities due to the possibility of clogging of filters leading to contamination of the clean room or affecting the biological processes in the bioreactor. When the product of bioreactor is a highly hazardous product, there is a concern for the safety of the operators from the exhaust gas.

There are also instances, where a drug or vaccine may need to be manufactured in an emergency and in those instances the requirement for clean room environmental controls can impede development and manufacturing. To allow a bioreactor to operate in an ISO 14644 Class 9 environment, it must have key features that will completely seal the bioreactor from the environment at every step of the operation of the bioreactor. Once assured that there cannot be any contact between the environment and the contents of a bioreactor, it will be possible to operate these bioreactors in ISO 14644 Class 9 environment. The present invention solves this problem.

Additionally, some bioreactor operations require a supply of nutrition that includes oxygen or pure oxygen pumped into bioreactors. While oxygen has considerable solubility in aqueous media, the high temperature of bioreactors keeps the solubility of oxygen low and a significant portion of it is exhausted it out of the bioreactor, adding substantial cost to the bioreactor operation due to large volume requirements.

Alternately, in some bioreactor operations an appropriate tension of carbon dioxide must be maintained, such as in the cultivation of Chinese Hamster Ovary cells to produce monoclonal antibodies and cytokines. This requires a continuous supply of carbon dioxide since the exhaust air continuously removes carbon dioxide. In a recirculating system of the present invention, the tension of carbon dioxide can be adjusted in the exhaust air to an optimal level, reducing the need to supplement the bioreactor with carbon dioxide.

In all instances where exhaust gas from a bioreactor is allowed to escape into the environment, it carries with it a substantial amount of moisture due to the heated content of the bioreactor. This causes evaporation of the content of bioreactor, leading to potential variation in the volume of bioreactor content, an undesirable effect that can alter the concentration of nutrients, essential growth factors and catalysts added to promote the biological reactions in the bioreactor.

The problems currently faced in the operations of bioreactors as enumerated above can be resolved by the present invention that allows the bioreactor to operate in a closed loop with several distinct advantages, such as no loss of oxygen, optimization of carbon dioxide tension, no loss of moisture from the bioreactor, no contamination of environment, improved control of conditions of bioreactor culture media, ability to operate in ISO 1400 9 environment, substantially reduced cost of operations, improved and consistent quality of products produced in the bioreactor, portability, and use of bioreactors in the field for on-demand production of products using biological entities.

### SUMMARY OF THE INVENTION

The present invention provides a design of a bioreactor exhaust assembly that can be connected to any type of bioreactor designed for growing all types of cells and organisms in an environment that does not need clean room level of environmental control (ISO 14644 Class 9). A closed system is provided wherein the exhaust gases are returned to the bioreactor after reducing or removing carbon dioxide from the exhaust.

The bioreactor exhaust recirculating apparatus comprises a bioreactor; a condenser capable of removing a gaseous element, such as carbon dioxide, from an exhaust gas of the bioreactor; and a return line extending from the condenser to the bioreactor capable of returning the remaining exhaust gas to the bioreactor. The bioreactor exhaust recirculating apparatus may further comprise a carbon dioxide sensor placed in the path of the exhaust gas.

The bioreactor may further comprise a cooling element, such as a refrigeration unit or a liquefied gas in contact with the condenser, capable of maintaining the temperature of condenser below the freezing temperature of the gaseous element to be removed. The liquefied gas may be, e.g., nitrogen or oxygen. The refrigeration unit may be capable of maintaining the temperature of the condenser below −75° C.

The bioreactor may further comprise culture media in the bioreactor, wherein one end of the bioreactor exhaust system comprises a return placed below the surface of culture media in the bioreactor.

The bioreactor exhaust recirculating apparatus may further comprise a heating element capable of heating the exhaust gas return to approximately the temperature of the culture media in the bioreactor.

The bioreactor exhaust recirculating apparatus may further comprise a filter capable of sterilizing the exhaust gas before returning it to the bioreactor.

The bioreactor exhaust recirculating apparatus may comprise a plurality of condenser elements capable of removing a gaseous element, such as carbon dioxide, from an exhaust gas of the bioreactor and a return from the condenser capable of returning the remaining exhaust gas to the bioreactor. The plurality of condensers may be arranged in parallel or in a series.

When bioreactor exhaust recirculating apparatus comprises a plurality of condenser elements, the exhaust gas may be diverted to a second condenser when a first condenser becomes blocked with a frozen gaseous element, such as carbon dioxide.

The present invention also includes an embodiment comprising a method of returning an exhaust gas to a bioreactor. The apparatus comprises a bioreactor according to the invention described above. The method comprises adding a cell to the culture medium capable of production of a protein; operating the bioreactor at a temperature suitable for production of the protein; passing the exhaust gas through the condenser and cooling the condenser to a temperature capable of freezing a gaseous element to be removed; heating the remaining gas exhaust to the temperature of the bioreactor; filter sterilizing the remaining gas exhaust; and returning the remaining gas exhaust to the bioreactor.

### BRIEF DESCRIPTION OF THE DRAWINGS

FIG. 1 is a side view of a bioreactor with an exhaust assembly.

FIG. 2 is a detailed view of the bioreactor exhaust assembly with a single condenser.

FIG. 3 is a detailed view of the bioreactor exhaust assembly having two condenser units.

### DETAILED DESCRIPTION OF INVENTION

Bioreactors are most often operated in an environmentally controlled environment, such as a cleanroom. A cleanroom has a controlled level of contamination that is specified by the number of particles per cubic meter at a specified particle size. To give perspective, the ambient air outside in a typical urban environment contains 35,000,000 particles per cubic meter in the size range of about 0.5 μm and larger in diameter, corresponding to an ISO 9 cleanroom, while an ISO 1 cleanroom allows no particles in that size range and only 12 particles per cubic meter of 0.3 μm and smaller. Cleanrooms are important in manufacturing of biological products, which are required to be free from microbial and particulate contamination and protected from moisture. Such products are manufactured and processed in these clean rooms, according to standards and guidelines issued by the FDA or other global regulatory institutions. Bioreactors are generally operated in at least ISO 14664 Class 8 environment that is expensive to construct and operate. A closed bioreactor wherein all operations are conducted in the vessel supporting nutrient medium can be used in less controlled or ISO 14644 Class 9 environment that is much cheaper to build and maintain.

Bioreactors require an adequate amount of nutrients along with required gases such as oxygen and carbon dioxide. There are two types of reactions mostly encountered in drug manufacturing, the one where the cells or organisms produce carbon dioxide in the process of their growth, such as bacteria, or where cells that require carbon dioxide as a source of carbon, such as mammalian cells.

Where an organism requires oxygen, the end product is often carbon dioxide and that has presented significant challenges to the design of disposable bioreactors for bacterial fermentation since it requires passing a very large volume of air or oxygen through the nutrient media. This supply of air/oxygen is partly to provide oxygen and partly to remove carbon dioxide (which dissolves in a nutrient medium) that is detrimental to the growth of bacteria. High levels of carbon dioxide in the culture media lowers the pH, which is also detrimental to the growth of bacteria. The room air generally contains about 0.035% of carbon dioxide whereas the exhaled human air has about 5% carbon dioxide. Exposure of healthy individuals for prolonged periods of 1.5 percent carbon dioxide will cause mild problems while exposure to 7 percent to 10 percent carbon dioxide will produce unconsciousness within a few minutes. This is because, at higher concentrations, carbon dioxide can displace the oxygen in the air. Because of this concern, carbon dioxide is called a “simple asphyxiant,” requiring the regulatory standards for requiring ventilation of manufacturing facilities. For example, ASHRAE 62 (American Society of Heating, Refrigerating and Air-Conditioning Engineers) requires fresh air inlet of at least 0.12 CFM per square foot of floor space.

The composition of air includes: Nitrogen 78.084%, Oxygen 20.9476%, Argon 0.934%, Carbon Dioxide 0.0314%, Neon 0.001818%, Methane 0.0002%, Helium 0.000524%. Krypton, 0.000114%, Hydrogen 0.00005%, Ozone 0.000007%, Xenon 0.0000087%, Nitrogen Dioxide 0.000002%, Iodine 0.000001%, Carbon Monoxide, Ammonia as trace. [Reference: CRC Handbook of Chemistry and Physics, edited by David R. Lide, 1997.] All of these gases have different freezing points. While air freezes at about −195° C., nitrogen freezes at −210° C., carbon dioxide at 78° C., and oxygen at 218.8° C. This difference in the freezing temperature of carbon dioxide provides an opportunity to extract carbon dioxide alone by adjusting the temperature of a gas.

In one embodiment, the exhaust gases are passed through a condenser that is kept cold to a temperature below the freezing temperature of carbon dioxide by such means as continuously keeping the condenser cold with passage of cold liquids in tube wrapped around the condenser.

In another embodiment, the invention allows the exhaust gas from which carbon dioxide has been removed to be heated prior to returning to the bioreactor preventing any temperature shock to the contents of the bioreactor.

In another embodiment, the present invention allows for the removal of condensed carbon dioxide from the condenser by alternating the passage of exhaust gas between a fouled condenser and a clean condenser while the fouled condenser is cleaned by raising its temperature to allow carbon dioxide to sublimate into atmosphere.

In another embodiment, the present invention allows for the reduction in the tension of carbon dioxide to a pre-determined level by selecting to pass only a portion of the exhaust through the condenser.

In another embodiment, the present invention conserves the essential components of the exhaust gases by returning them to the bioreactor, reducing the need for large volumes of gases required for the operation of the bioreactor. This substantially reduces the quantity of oxygen gas or carbon dioxide required to operate the bioreactor since exhaust gases remove a large quantity of undissolved oxygen and carbon dioxide in the culture media.

In another embodiment, the present invention allows operation of the bioreactor in ISO 9 class environment. Whereas standard bioreactor operations exhaust the effluent gases to the environment, the open end of the exhaust creates a permanent risk for contamination of the contents of the bioreactor; it is more important to realize this when using flexible disposable containers as bioreactors, where it is a likely possibility that the air from the room may be sucked into the container. Exhausting gases from bioreactor provides contamination possibilities that may require the use of controlled environmental conditions. One solution to resolving this problem is to eliminate all exhausts in a bioreactor. Instead, the gases in the bioreactor are recirculated according to the present invention and when excess carbon dioxide is produced in the nutrient medium, reducing its tension in the exhaust gas prior to recirculating the exhaust gas back into the bioreactor.

The present invention includes a method for removing carbon dioxide from the effluent gases produced in the bioreactor by installing a plurality of condensers kept cold at a temperature to cause freezing of carbon dioxide as the exhaust gases pass through the condensers.

Oxygen tension in the nutrient medium is important for many bioreactions, and this is controlled in the present invention by recirculating the exhausted oxygen, substantially reducing the quantity of oxygen required to operate the bioreactor.

Carbon dioxide tension in the nutrient medium is important for many bioreactor reactions, and this is controlled in the present invention by recirculating the exhaust gas from which at least part of carbon dioxide is removed by freezing.

The present invention provides an ideal solution for expanding the Use of bioreactors and making them more affordable. This is of particular value when manufacture drugs and vaccines in emergencies, developing new drugs, manufacturing clinical test supplies and generally producing commercial scale biological drugs at a lower cost. The last consideration has recently become very important, as generic biological drugs called biosimilars have begun appearing in the market following the expiry of originator patents on these drugs. The biosimilars must be able to manufacture these molecules at a cheaper cost to compete, and this invention fills that need.

Protein-based drugs are frequently derived from biological cultures including human and animal cells, bacteria, yeast, plant cells, hybridomas and any such biological compositions that are capable of replicating and undergoing metabolism. Additionally, biological entities are grown into organs and products of cell and gene therapy. These protein-based drugs are routinely produced using traditional bioreactors and more recently disposable bioreactors.

In one aspect, the present invention provides a bioreactor suitable for preparing a biological product while operating in an ISO 14664 Class 9 environment using a bioreactor wherein the risk of contaminating the content of bioreactor from the environment and contaminating the environment from the content of bioreactor are minimized by keeping the exhaust gases from escaping to environment and thereby also keeping close the connectivity between the environment and the bioreactor. More particularly, the invention provides an apparatus and method, which is universal in the sense that the apparatus may be used to prepare a biological product regardless of the nature of the biological culture.

The bioreactor may be any type capable of producing a biological product. The dimensions and shape of the bioreactor can be varied to suit the needs of the user based on, expense or the nature of the biological culture to be used, suppliers available, etc. Along with gas exchange and mixing, other criteria to selecting a container shape include shear force and cell yield. A two-dimensional container that is longer than wider is preferred. A wide range of interior volumes is contemplated, ranging from 1 L to 10000 L. The containers useful in the various embodiments of the invention may be of any size suitable for containing a liquid. For example, the container may have a volume between 1-40 L, 40-100 L, 100-200 L, 200-300 L, 300-500 L, 500-750 L, 750-1,000 L, 1,000-2,000 L, 2,000-5,000 L, or 5,000-10,000 L. In some instances, the container has a volume greater than 1 L, or in other instances, greater than 10 L, 20 L, 40 L, 100 L, 200 L, 500 L, or 1,000 L. Volumes greater than 10,000 L are also possible. Preferably, the container volume will range between about 1 L and 1000 L, and more preferably between about 10 L and 500 L, and even more preferably between 25 L and 100 L. Preferably, the containers have an interior volume of 10 L-100 L.

Although a rectangular shape is illustrated in the figures, other shapes can also be advantageous. These shapes include, but are not limited to, an oval, a cylinder, a cylindrical tube or a cuboid. It should also be understood that many of the components described herein also are desirably flexible, e.g., the containers desirably comprise flexible biocompatible polymer containers. The flexible bioreactor may be, e.g., silicone, polycarbonate, polyethylene, and polypropylene. Non-limiting examples of flexible materials include polymers such as polyethylene (e.g., linear low density polyethylene and ultra-low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics.

The containers may have any thickness suitable for retaining the culture medium within, and may be designed to have a certain resistance to puncturing during operation or while being handled. For example, the walls of a container may have a total thickness of less than or equal to 250 mils (1 mil is 25.4 micrometers), less than or equal to 200 mils, less than or equal to 100 mils, less than or equal to 70 mils, less than or equal to 50 mils, less than or equal to 25 mils, less than or equal to 15 mils, or less than or equal to 10 mils. In certain embodiments, the container may include more than one layer of material that may be laminated together or otherwise attached to one another to impart certain properties to the container. For instance, one layer may be formed of a material that is substantially oxygen impermeable. Another layer may be formed of a material to impart strength to the container. Yet another layer may be included to impart chemical resistance to the fluid that may be contained in the container.

In addition, the bioreactor preferably is seamless in order to improve its strength and avoid deposition of growing cells in the media. All or portions of the bioreactor also are desirably translucent, or more desirably transparent, to allow viewing of contents of the container. The latter is preferred when it is desirable to irradiate the culture medium within the bioreactor.

Sheets of plastic may be used to form three-dimensional containers by making more than two walls, or incorporating pleats or darts in a two walled structure. Other characteristics of the material used for making the container may be important in culturing various types of cells or microorganisms. The inside of the container may be treated with substances that increase or decrease cell adhesion, depending on the culture conditions desired and the kind of cells.

In a preferred embodiment, the container is constructed with welded seams. Other means to seal the seams of the container include, but are not limited to, heat, ultrasound and radio wave welding. Other kinds of plastic may lend themselves to other kinds of manufacture, such as mold injection.

The container comprises one or more ports to serve as inlets or outlets for gases or liquids. The ports are constructed of rigid or semi-rigid materials that are compatible with the material used for construction of the container. In preferred embodiments, any standard plastic tubing or molded plastic can be used to construct the ports, and they are welded into the seams of the container according to standard techniques. A variety of such ports is commercially available, e.g., for use in single-use bags and similar containers. The structures that form the ports can also be varied, e.g., to accommodate different volumes of media, autoclave techniques and port functions. These ports may have any of the port variations known in the art, including but not limited to, Luer-lock fittings, rubber sheet gaskets, and frangible valves.

The disposable bioreactors of the present invention preferably are pre-sterilized. Various sterilization techniques may be used. The choice of technique is partially dependent on the type of plastic chosen (Lee et al., 1995, in Handbook of Polymeric Biomaterials, CRC Press, Boca Raton, p 581-597). Sterilization techniques that are common in the art include, but are not limited to, dry heat, autoclaving, radiation, and ethylene oxide gas.

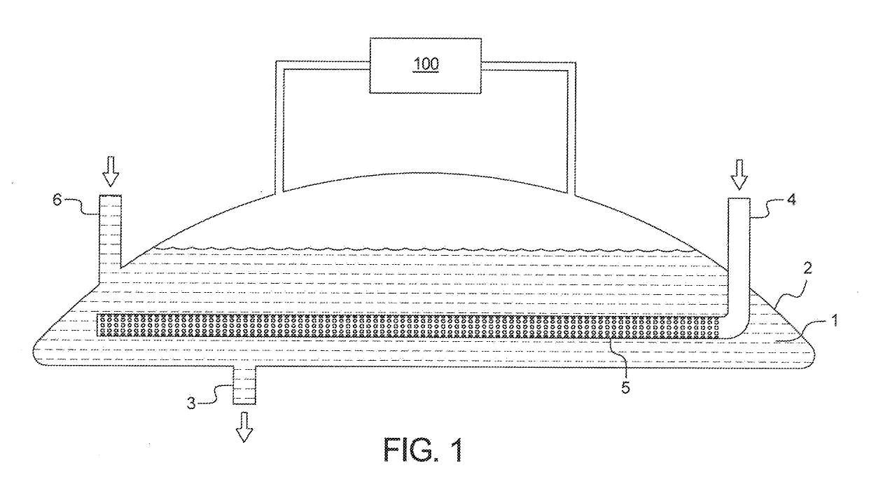

Turning initially to FIG. 1, wherein is illustrated a bioreactor 2, the nutrient media in the bioreactor 1, having a port for sampling and/or removing liquid 3, a gas inlet 4 and sparging system 5 and a liquid inlet port 6. Attached to the bioreactor is the bioreactor exhaust gas recirculating apparatus 100.

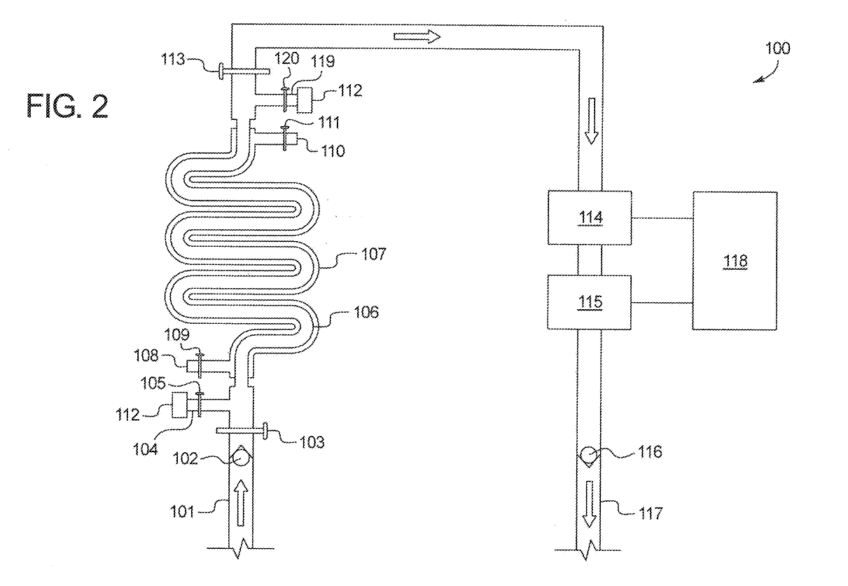

FIG. 2 illustrates the bioreactor exhaust gas recirculating apparatus 100 in more detail. There are three portions delineated by valves 103 and 113. The first portion comprises 101 to 103. The second portion comprises 104 to 112. And the third portion comprises 113 to 118. In the first portion, an exhaust tube 101 vents the exhaust gases from the bioreactor. A directional one-way valve to route exhaust gas from the bioreactor 102 prevents the exhaust gas from reentering the bioreactor through this tube. An exhaust control valve 103 is used when the condenser must be cleared. The second portion of the apparatus is designed to remove at least one gaseous element from the exhaust gas by passing the gas through a coiled condenser 106 that is jacketed by a refrigerating unit 107 capable of freezing the gaseous element. In FIG. 2 this is accomplished by passing cooling liquid from the inlet for liquid coolant 108 through 107 to the outlet for liquid coolant 110. Valves 109 and 111 can be adjusted to control the flow through 107. There are other ways of cooling the exhaust gas not illustrated here. The gaseous portion of the exhaust gas leaves the condenser through the gas tube 117. The third portion of the bioreactor exhaust recirculating system includes the elements necessary to return the exhaust gas to the bioreactor without disrupting or harming the biological culture in the bioreactor. Elements 114 and 115 can be in either order, but preferably in the order shown. The heater 114 raises the temperature of the exhaust gas to an acceptable level to avoid killing or slowing down the production of the cell culture, preferably to the temperature of the culture. Element 115 is a sensor, such as a CO2 sensor, to monitor the level of the gaseous element removed by the condenser. The entry of exhaust gas into liquid media in bioreactor passes through a one-way valve 116. When the amount of frozen gaseous element impedes the flow of gas through the exhaust system, valves 103 and 113 are closed. In addition, the coolant is turned off by closing valves 109 and/or 111. Valves 105 and 120 are opened, and the air is passed through a sterile filter 112 into the inlet port 104, warming the frozen gaseous element that then passes out of outlet tube 119. Once the condenser has been cleared of CO2, valves 105 and 120 are closed, and valves 103 and 113 are reopened allowing the exhaust system to continue to remove undesired CO2.

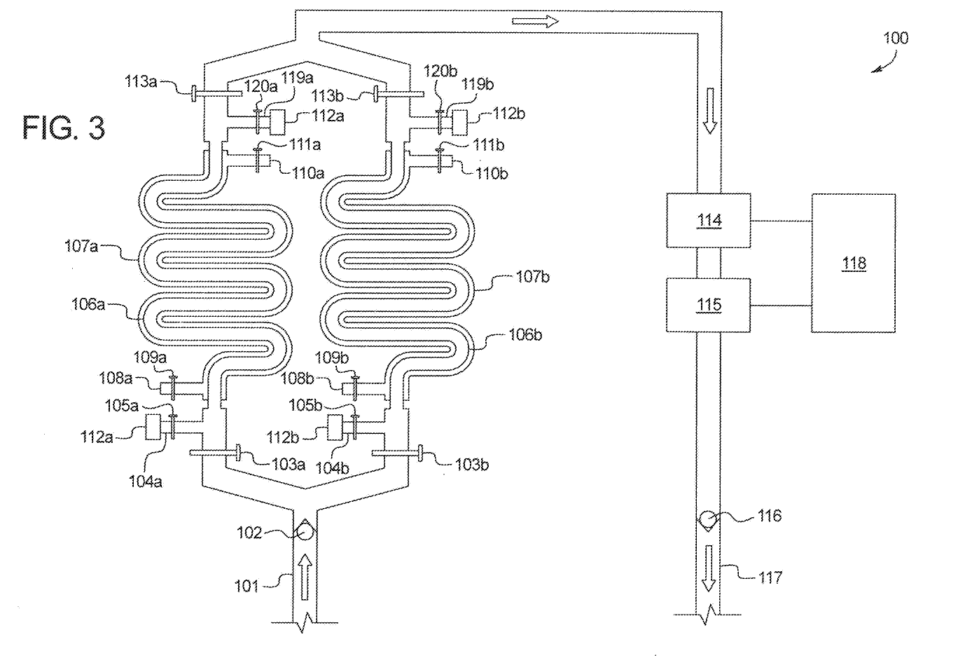

FIG. 3 illustrates the bioreactor exhaust gas recirculating apparatus 100 wherein there are two condensers. This embodiment allows one to switch from one condenser to the other when one condenser becomes blocked by frozen gaseous element. Normal operation is to have only one condenser operating at a time. Valves 103b and 113b would be closed, and 103a and 113a would be open. When the first condenser becomes blocked, valves 103a and 113a are closed, and valves 103b and 113b are opened. Removal of the frozen gaseous element would proceed as described above while the exhaust from the bioreactor is diverted to the second condenser system. When the second condenser system becomes blocked, the bioreactor exhaust is diverted back to the first condenser and the second condenser is cleared.

As previously mentioned, the containers desirably include one or more sensors or probes for monitoring one or more process parameters inside the containers such as, for example, cell density, temperature, pressure, pH, dissolved oxygen (DO), dissolved carbon dioxide (DCO₂), mixing rate, and gas flow rate. The sensors for DO, and DCO₂ are desirable optical sensors, with the first two more desirably being disposable (e.g., TruFluor sensors, Finesse Solutions LLC, Santa Clara, Calif. or CellPhase sensors, Fluorometrix Corporation, Stow, Mass. 01775). Each sensor is intended to be in communication with a computer-implemented control system (e.g., a computer) for calculation and control of various parameters and for display and user interface. Such a control system may also include a combination of electronic, mechanical, and/or pneumatic systems to control the aforementioned processing parameters as required to stabilize or control the parameters (e.g., pH may be adjusted by the addition of CO₂ or ammonia). It should be appreciated that the control system may perform other functions, and the invention is not limited to having any particular function or set of functions.

The one or more control systems described herein can be implemented in numerous ways, such as with dedicated hardware and/or firmware, using a processor that is programmed using microcode or software to perform the functions recited above or any suitable combination of the foregoing. More specifically, the control system(s) 118 can control the flow of the cooling liquid through 107 by adjusting the valves at 109 and 111; control the opening and closing of valve 103, and 113; control of the valves 105 and 120 associated with clearing the condenser; control of the heater 114; and/or use the information received from the sensor 115 to adjust the level of cooling in the condenser.

Alternately, the invention may be devoid of any sensors and the measurements of the characteristics of culture media made on the sample collected through the port 3, which is also used to introduce biological culture in the container 2 and to remove the nutrient media at the end of the bioreaction cycle. It is noteworthy that port 3 requires an extension long enough to allow delivery of nutrient media for testing or further processing to a controlled environment, e.g., ISO 14644 Class 7 or lower.

The bioreactor described herein is useful in the production of a biological product from a nutrient medium of a predetermined volume. In a related embodiment, the invention provides a method which contemplates providing a bioreactor as herein described, filling each container with nutrient medium; activating the selectively sparging filter and heating or cooling elements, adding biological culture and allowing the bioreactor to stand for a specific time, detecting the density of cells in the nutrient medium in a container at predetermined time intervals; and removing the nutrient medium and the biological product produced thereby from the bioreactor when the density of the cells in the nutrient medium in the container reaches a predetermined value. The exhaust gases are recirculated through the container while replenished with appropriate quantities of oxygen or carbon dioxide; when carbon dioxide is produced in a bioreaction, a carbon dioxide scrubber is provided to remove carbon dioxide from the exhaust gases.

One of the advantages of the inventive bioreactor and related method is the reduced cost of environmental control required to operate bioreactors. The present invention can be operated in an ISO 14644 Class 9 environment as opposed to Class 8 environment generally required for these operations.

The relatively lower amount of gas introduced (relative to known processes) has the further advantage of generating less turbulence and foam in the headspace of the container.

Each container is provided with a submicron filter that assists in maintaining sterility of the nutrient medium and gases introduced. The filter may be of any suitable size and porosity, but is preferably an HEPA filter, having an average porosity of from about 0.3 μm to about 0.1 μm, and more preferably of about 0.22 μm.

Generally, the invention provides bioreactors and methods, which are universal in the sense that the invention is suitable and adaptable for processing a variety of compositions, including both biologic and non-biologic components. Indeed, an inventive bioreactor designed for use with mammalian cells, for example, may be used for culturing bacteria, allowing ease of manufacturing.

The present invention is not limited to the embodiments described and exemplified above but is capable of variation and modification without departure from the scope of the appended claims.

1. A bioreactor exhaust recirculating apparatus comprising:
a. a bioreactor;
b. a condenser capable of removing a gaseous element from an exhaust gas of the bioreactor; and
c. a return line extending from the condenser to the bioreactor capable of returning the remaining exhaust gas to the bioreactor.

2. The apparatus of claim 1, wherein the gaseous element to be removed is carbon dioxide.

3. The apparatus of claim 1, further comprising a cooling element capable of maintaining the temperature of condenser below the freezing temperature of the gaseous element to be removed.

4. The apparatus of claim 3, wherein the cooling element is a refrigeration unit.

5. The apparatus of claim 3, wherein the cooling element comprises a liquefied gas in contact with the condenser.

6. The apparatus of claim 3, wherein the cooling element is capable of maintaining the temperature of condenser below −75° C.

7. The apparatus of claim 5, wherein the liquefied gas is selected from nitrogen and oxygen.

8. The apparatus of claim 1, further comprising culture media in the bioreactor, wherein the return line end is placed below the surface of culture media in the bioreactor.

9. The apparatus of claim 1, further comprising a heating element capable of heating the exhaust gas return to approximately the temperature of the culture media in the bioreactor.

10. The apparatus of claim 1, further comprising a filter capable of sterilizing the exhaust gas.

11. A bioreactor exhaust recirculating apparatus comprising:
a. a bioreactor;
b. a plurality of condenser elements capable of removing a gaseous element from an exhaust gas of the bioreactor; and
c. a return line from the condenser capable of returning the remaining exhaust gas to the bioreactor.

12. The apparatus of claim 11, wherein a plurality of condensers are arranged in parallel or in a series.

13. The apparatus of claim 12, wherein the apparatus comprises two condensers in parallel.

14. The apparatus of claim 11, further comprising a cooling element capable of maintaining the temperature of condenser below −75° C., wherein the gaseous element is carbon dioxide.

15. The apparatus of claim 14, further comprising a carbon dioxide sensor placed in the path of the exhaust gas.

16. A bioreactor exhaust recirculating apparatus comprising:
a. a bioreactor; and
b. an exhaust recirculating apparatus comprised of three connected portions, wherein a first portion is in fluid connection with a second portion, and the second portion is in fluid connection with a third portion;
i. wherein the first portion comprises: (1) an exhaust tube connected to the bioreactor having a one-way valve inside the exhaust tube directed away from the bioreactor and (2) a valve capable of closing the connection between the first portion and the second portion;
ii. wherein the second portion comprises: (1) a condenser comprising a coiled tube wrapped by a second tube external to the flow of exhaust gas, wherein the second tube is capable of delivering a cooling agent capable of freezing the gaseous element or a warming agent capable of thawing the frozen gaseous element and wherein the condenser is in fluid connection with the first portion and third portion; (2) an inlet port and an outlet port capable of introducing a gas into the condenser wherein each port comprises a valve for opening or closing the port, each port optionally having a filter capable of sterilizing the gas being introduced or exiting the condenser, and (3) a valve for closing the connection between the second portion and the third portion; and
iii. wherein the third portion comprises: (1) a return tube extending from the condenser to the bioreactor capable of returning the remaining exhaust gas to the bioreactor; (2) a heater element, (3) a sensor, and (4) a one-way valve inside the return tube directed toward the bioreactor.

17. The apparatus of claim 1 or claim 11 or claim 16, further comprising one or more control elements capable of automated opening and closing of valves and following programmed instructions to direct the flow of exhaust gas.

18. A method of removing a gaseous element from the exhaust gas of a bioreactor comprising;
a. providing a bioreactor;
b. attaching to the bioreactor a recirculating bioreactor exhaust apparatus comprising:
i. a first portion of a recirculating bioreactor exhaust apparatus comprising: (1) an exhaust tube connected to the bioreactor having a one-way valve inside the tube directed away from the bioreactor and (2) a valve capable of closing the connection between the first portion and a second portion;
ii. a second portion of a recirculating bioreactor exhaust apparatus comprising: (1) a condenser capable of removing a gaseous element from an exhaust gas of the bioreactor; (2) a cooling element external to the condenser capable of maintaining the temperature of condenser below the freezing temperature of the gaseous element to be removed; (3) an inlet port below the condenser and an outlet port above the condenser for removing the frozen gaseous element, each port comprising a valve for opening and closing the port; and (4) a valve for closing the connection between the second portion and a third portion;
iii. a third portion of a recirculating bioreactor exhaust apparatus comprising: (1) a return line from the condenser to the bioreactor capable of returning the remaining exhaust gas to the bioreactor; (2) a heating element; (3) a sensor; and (4) a one-way valve inside the return tube directed toward the bioreactor;
c. adding a culture medium to the bioreactor;
d. adding a cell to the culture medium;
e. operating the bioreactor at a temperature suitable for growth of the cell culture;
f. allowing the exhaust gas to pass through the condenser;
g. cooling the condenser to a temperature capable of freezing the gaseous element to be removed;
h. heating the remaining exhaust gas leaving the condenser to the temperature of the bioreactor;
i. returning the remaining exhaust gas to the bioreactor;
j. monitoring the sensor in the third portion for the presence of the gaseous element being removed by the condenser;
k. when the level of gaseous element being removed by the condenser exceeds a predetermined value, the condenser is cleared to remove the gaseous element from the condenser.

19. The method of claim 18, wherein step (k) comprises the steps of:
a. closing the valve between the first portion and the second portion;
b. closing the valve between the second portion and the third portion;
c. attaching a filter to the inlet port of the second portion and opening its valve;
d. optionally attaching a filter to the outlet port of the second portion and opening its valve;
e. introducing a warming agent such as compressed air;
f. optionally measuring the gas leaving the outlet port for the gaseous element;
g. closing the inlet and outlet ports;
h. opening the valve between the first portion and the second portion;
i. opening the valve between the second portion and the third portion;
j. resuming the flow of exhaust gas from the bioreactor through the recirculating bioreactor exhaust apparatus.

20. The method of claim 18, wherein a second condenser is added to the recirculating bioreactor exhaust apparatus.

21. The method of claim 20, wherein step (k) comprises the steps of:
a. closing the valve between the first portion and the first condenser;
b. closing the valve between the first condenser and the third portion;
c. opening the valve between the first portion and the second condenser;
d. opening the valve between the second condenser and the third portion;
e. attaching a filter to the inlet port of the first condenser and opening its valve;
f. optionally attaching a filter to the outlet port of the first condenser and opening its valve;
g. introducing a warming agent such as compressed air;
h. optionally measuring the gas leaving the outlet port for the gaseous element;
i. closing the inlet and outlet ports of the first condenser;
j. opening the valve between the first portion and the first condenser;
k. opening the valve between the first condenser and the third portion;
l. closing the valve between the first portion and the second condenser;
m. closing the valve between the second condenser and the third portion;
n. attaching a filter to the inlet port of the second condenser and opening its valve;
o. optionally attaching a filter to the outlet port of the second condenser and opening its valve;
p. introducing a warming agent such as compressed air;
q. optionally measuring the gas leaving the outlet port for the gaseous element;
r. closing the inlet and outlet ports to the second condenser; and
s. repeating steps a-r.
